(19) United States
(12) Patent Application Publication
(10) Pub. No.: US 2006/0257879 A1
(43) Pub. Date: Nov. 16, 2006

(54) Methods and kits for detecting an enzyme capable of modifying a nucleic acid

(76) Inventors: Stuart Wilson, London (GB); Christopher John Stanley, Cambridge (GB); Sharon Banin, London (GB)

Correspondence Address: WOLF GREENFIELD & SACKS, PC, FEDERAL RESERVE PLAZA, 600 ATLANTIC AVENUE, BOSTON, MA, 02210-2206, US

(21) Appl. No.: 10/566,792

(22) PCT Filed: Aug. 2, 2004
(86) PCT No.: PCT/GB04/03325

§ 371 Date: Jun. 2, 2006

(30) Foreign Application Priority Data
Aug. 1, 2003 (GB) 0318110.4

(51) Int. Cl. C12Q 1/68; C12Q 1/34

(52) U.S. Cl. 435/6; 435/18

(57) ABSTRACT

The present invention provides a method of detecting an enzyme in a sample wherein the enzyme is capable of adding or removing a chemical moiety to or from a nucleic acid molecule, which thereby confers altered sensitivity of the nucleic acid molecule in a subsequent process. The invention also relates to diagnostic methods which take advantage of the method of the invention and kits which are useful for carrying out the method of the invention.

### FIELD OF THE INVENTION

The invention relates to methods and kits for detecting an enzyme in a sample which is capable of modifying a nucleic acid molecule by detecting the change in the nucleic acid molecule caused by the enzyme.

### BACKGROUND TO THE INVENTION

Sensitive methods exist to detect target molecules such as particular nucleic acids, proteins or more simple molecules. The presence of such molecules may be used to indicate an on-going infection or environmental contamination, for example. In prion diseases it would be useful to be able to detect the prion protein where no nucleic acid is present. Also, at certain stages of a viral infection there will be virus antigen present but little viral nucleic acid present. Here it will be useful to be able to detect the viral antigen directly. In order for these methods to be very sensitive and to detect as little as a single molecule the methods must also have high specificity. This high specificity is often achieved by binding two reporters to the target molecule that is to be detected.

In the case of the highly sensitive polymerase chain reaction (PCR), for example, two short nucleic acid probes or primers recognise the target nucleic acid. The detection of the target nucleic acid is thus only achieved when both primers are bound to, and linked through, the same target molecule. Non-specific interactions of the primers with other molecules are not detected unless both primers bind to and are linked by this non-specific interaction. The conditions of the reaction are such that the latter is highly unlikely. The PCR method and other molecular amplification methods, well known in the art, such as Nucleic acid sequence-based amplification (NASBA; Compton, 1991)(1), Transcription Mediated Amplification (TMA; Gen-probe, Inc.) and Self-sustained sequence replication (3SR; Fahy et al., 1991)(2) can be used to detect target nucleic acids.

Immunoassays are often employed in order to detect specific analytes/antigens of interest. Here an antibody, usually a monoclonal antibody, is used in order to allow specific detection of the analyte/antigen. Immuno detection methods can be broadly split into two main categories; solution-based techniques such as enzyme-linked immunosorbent assays (ELISA), immunoprecipitation and immunodiffusion, and procedures such as Western blotting and dot blotting where the samples have been immobilized on a solid support.

Western blot analysis relies on a primary antibody directed against the antigen/analyte, which is added to a membrane containing immobilized antigen/analyte to allow binding to potential antigenic sites. Next, a secondary antibody-enzyme conjugate which recognizes the primary antibody is added in order to find locations where the primary antibody bound. The enzyme, commonly alkaline phosphatase or horseradish peroxidase, conjugated to the secondary antibody can catalyze a reaction with a chemiluminescent substrate in the third step leading to emission of light from the membrane at the reaction site. An x-ray film exposed to the signal provides a visual indication of potential primary antibody recognition. The action of horseradish peroxidase or alkaline phosphatase on a chemiluminescent substrate can give sensitivity down to the picomolar range. Antigens/analytes can be immobilized on nitrocellulose or polyvinylidene fluoride (PVDF) membranes by numerous methods. The ability to detect a given antigen/analyte depends upon the amount of antigen per unit area of the membrane and on the characteristics of the primary antibody.

ELISAs provide sensitive and quantitative detection of specific antigens/analytes. The most common ELISAs are based on an antibody-sandwich format. A sandwich ELISA generally requires two antibodies that are directed against a particular antigen. One antigen is coated onto the wells of the ELISA plate. The wells are then “blocked” using a non specific protein solution (such as milk protein solution) to keep background levels down to a minimum. Samples containing the antigen in solution are then added to the wells and incubated for a sufficient amount of time to allow antigen binding to the immobilized antibody. The second antibody can then bind to the antigen to complete the “sandwich”. The second antibody is detected with an enzyme conjugate specific for the second antibody. As an alternative, the second antibody can be labeled itself to allow subsequent detection. When the enzyme substrate is added to the wells in the final step, the conjugated enzyme, which is linked to the antigen, is detected by observing a reaction product which may be calorimetric, fluorescent or chemiluminescent depending on the enzyme and substrate used, using an ELISA plate reader.

The most commonly employed enzymes in immunoassays are horseradish peroxidase (HRP) and alkaline phosphatase (AP). Such enzymes can react with a substrate chromogen to give a coloured product in the presence of an antigen. For example, a substrate chromogen commonly used in conjunction with alkaline phosphatase is 5-bromo, 4-chloro, 3-indolylphosphate (BCIP). An additive such as iodoblue tetrazolium (INT) may also be used to enhance the final colour of the precipitate at the reaction sites, that is where the primary and secondary antibodies have bound to the antigen (which would be a yellow-brown colour for BCIP with INT).

Alkaline phosphatase also has the ability to remove 5′ phosphate groups from DNA and RNA. It can also remove phosphates from nucleotides and proteins. These enzymes are most active at alkaline pH. Three major types are commonly employed in immunoassays. Bacterial alkaline phosphatase (BAP) is a highly active enzyme. Calf intestinal alkaline phosphatase (CIP) is purified from bovine intestine, and can be inactivated using protease digestion or heat, for example. Shrimp alkaline phosphatase is derived from a cold-water shrimp and can be inactivated using heat treatment fairly readily.

HRP can be used in a number of bioassays. Peroxidase activity is also present in many cells. Many fluorogenic substrates for HRP are well known in the art and are commercially available. One example is Amplex Red Reagent (Molecular Probes), 10-acetyl-3,7-dihydroxyphenoxazine, which can react with H₂O₂ in a 1:1 stochiometry in the presence of HRP to produce highly fluorescent resorufin. An alternative substrate is scopoletin, where HRP catalyzes conversion of the fluorescent scopoletin to a nonfluorescent product. Such substrates are commonly included in ELISA kits to allow detection of sites where an antigen/analyte is present.

Numerous attempts have been made to combine the advantages of immunoassays and nucleic acid amplification techniques. Indirect conjugation methods may be used to link a protein to a nucleic acid molecule. For example, an enzyme such as alkaline phosphatase may be covalently bound to a molecule such as biotin and digoxigenin. This conjugate in turn can then be non-covalently attached to a biotinylated nucleic acid probe via a streptavidin bridge, to be used, for example in Southern and Northern blotting techniques. Such methods can produce consistent results, however the protocols can take much longer than those of direct conjugation methods. Usually several incubation and washing steps are required to bind additional bridging molecules such as streptavidin or an antibody to the labeled probe before the enzyme and substrate can be introduced. Furthermore, with each additional step there is an increased chance of adding background to the signal.

Thus, direct conjugation of an enzyme to a probe is a preferable option, to increase speed and maximise sensitivity. Alkaline phosphatase-conjugated oligonucleotides (Sigma-Genosys) can be used for routine screening applications such as Southern (DNA) and Northern (RNA) blotting, gene mapping and restriction fragment length polymorphism (RFLP) analysis. They can also be used for in situ hybridizations.

Enzyme immunoassays have been established as the most ubiquitous methods for detection of antigen. They are simple, robust and easy to perform. In those cases where extra sensitivity is required more complex and expensive nucleic acid amplification tests such as the Polymerase Chain Reaction (PCR) can be performed. Numerous attempts have been made to combine the advantages of both approaches. For example, there is use for a sensitive nucleic acid test that can detect antigen. This would be useful in prion detection where there is no associated nucleic acid or in blood bank screening where, at certain times post-infection, there can be virus antigen but little viral nucleic acid.

Previous attempts to combine the immuno and nucleic acid approach by using antibodies labeled with nucleic acids (so-called immuno PCR) have had problems. Linking DNA to antibodies is problematical and the linked DNA is ‘sticky’ and any unbound DNA is not easily washed from the system prior to detection which can lead to non-specific binding and a high background in the assay.

The present invention overcomes the problems associated with prior art methods as described below.

### BRIEF DESCRIPTION OF THE INVENTION AND DEFINITION OF TERMS

The present invention seeks to provide improved methods for detecting an enzyme in a sample which is capable of modifying a nucleic acid molecule by detecting the change in the nucleic acid molecule caused by the enzyme.

According to a first aspect of the invention there is provided a method of detecting an enzyme in a sample wherein the enzyme is capable of adding or removing a chemical moiety to or from a nucleic acid molecule, which thereby confers altered sensitivity of the nucleic acid molecule in a subsequent process, the method comprising:

allowing the sample to be tested for the presence of the enzyme to interact with the nucleic acid molecule; and

testing for interaction of the enzyme with the nucleic acid molecule by detecting the altered sensitivity of the nucleic acid molecule caused by the enzyme.

The method relies on the fact that if the enzyme is present it will be able to add a chemical moiety to the nucleic acid molecule or remove a chemical moiety from the nucleic acid molecule. This moiety addition or removal alters the sensitivity of the nucleic acid molecule in a subsequent process. The increased or decreased sensitivity to the subsequent process may be detected, thereby allowing a determination of the presence of an enzyme in the sample under test.

The term “chemical moiety” is well known in the art and includes by way of example and not limitation, phosphate groups, carbohydrate groups, nucleotides and acetyl groups etc. Any “chemical moiety” is included within the scope of the invention provided its addition or removal to or from a nucleic acid molecule may be catalysed by an enzyme to alter the sensitivity of the nucleic acid molecule in a subsequent process.

The method is not limited to addition or removal of a single chemical moiety per nucleic acid molecule. The term “a chemical moiety” may, therefore, include multiple copies of the chemical moiety in question.

An “addition” of a chemical moiety may include, by way of example but not limitation, addition of new base pairs or acetyl or phosphate groups. Addition may be at the 5′ or 3′ end or at any point within the nucleic acid molecule.

A “removal” of a chemical moiety may include, but is not limited to, removal of bases and phosphate groups from terminal ends of the nucleic acid molecule or from anywhere along the nucleic acid molecule.

“Altered sensitivity” is defined herein to include any change in the behaviour or properties of the nucleic acid molecule when subjected to a further process as compared to the starting, unmodified (in terms of addition or removal of a chemical moiety) nucleic acid molecule.

Many such additions or removals of chemical moieties which confer altered sensitivity of the nucleic acid molecule in a subsequent process are well known in the art, but are not intended to be limiting with respect to the present invention. For example, the addition or removal of a chemical moiety to or from a nucleic acid molecule may enhance the susceptibility of that molecule to degradation. This may be, for example, by increasing susceptibility of the nucleic acid molecule to nuclease activity. The nuclease activity may be non-sequence specific, for example 5′-3′ or 3′-5′ processive exonuclease activity. Alternatively, it may be sequence specific. For example, the addition or removal of a chemical moiety to or from a nucleic acid molecule may introduce a new restriction endonuclease recognition site (or indeed remove a restriction endonuclease recognition site) into the nucleic acid molecule, which may be detected by utilising the specific restriction endonuclease which will be able to digest those nucleic acid molecules to which a chemical moiety has been added or removed but not those where no chemical moiety has been added or removed.

The nucleic acid molecules for use in the methods, and inclusion in the kits, of the invention, must be of sequence and structure such that the enzyme that is being detected in the sample may cause the addition or removal of a chemical moiety to or from the nucleic acid molecule, thereby conferring altered sensitivity on the nucleic acid molecule in a subsequent process. “Nucleic acid” is defined herein to include any natural nucleic acid and natural or synthetic analogues that are capable of being modified by the addition or removal of a chemical moiety to or from a nucleic acid molecule which thereby confers altered sensitivity on the nucleic acid molecule in a subsequent process. Suitable nucleic acid molecules may be composed of, for example, double or single-stranded DNA and double or single-stranded RNA. Nucleic acid molecules which are partially double-stranded and partially single-stranded are also contemplated, provided the enzyme activity being investigated may add or remove a chemical moiety to or from the nucleic acid molecule. Most preferably the nucleic acid molecules will comprise dsDNA. The term “nucleic acid” encompasses synthetic analogues which are capable of being modified by an enzyme in a sample in an analogous manner to natural nucleic acids, for example nucleic acid analogues incorporating non-natural or derivatized bases, or nucleic acid analogues having a modified backbone. In particular, the term “double-stranded DNA” or “dsDNA” is to be interpreted as encompassing dsDNA containing non-natural bases. Similarly, “dsRNA” is to be interpreted as encompassing dsRNA containing non-natural bases.

A “sample” in the context of the present invention is defined to include any sample in which it is desirable to test for the presence of a particular enzyme. Thus the sample may be a clinical sample, or an in vitro assay system for example. The sample may comprise tissue or cells for example.

“Diagnosis” is defined herein to include monitoring the state and progression of the disease, checking for recurrence of disease following treatment and monitoring the success of a particular treatment. The tests may also have prognostic value, and this is included within the definition of the term “diagnosis”. The prognostic value of the tests may be used as a marker of potential susceptibility to disease associated with elevated phosphatase levels. Thus patients at risk may be identified before the disease has a chance to manifest itself in terms of symptoms identifiable in the patient.

### ADVANTAGES AND APPLICATIONS OF THE INVENTION

The advantages of the present invention include avoiding the use of the ‘sticky’ DNA-antibody conjugates—in fact the same alkaline phosphatase conjugates can be used that have already been optimized and characterized for many immuno applications. In addition, in the assay there is no need to wash away the DNA as the DNA is used as the target and can only be detected when it has been modified. An additional advantage is that immuno PCR can only amplify each DNA target that remains bound to the antigen through the antibody. In the invention described herein, however, the DNA is used as a substrate for antibody bound alkaline phosphatase and each molecule of phosphatase will generate many molecules of detectable DNA target. Thus prior to PCR there has already been an amplification of the DNA target to be detected. This method of two rounds of amplification; one by the antibody-bound enzyme and the second by a nucleic acid amplification method such as PCR gives a much increased sensitivity over that of traditional immuno PCR. There is, therefore, an advantage in providing a method of linking immunoassays and nucleic acid amplification techniques in order to increase the sensitivity of an immunoassay.

Another application of this technique is in the detection of free phosphatase that is important in relation to infectious or non-infectious disease. In infectious diseases, for example, most bacteria or fungi contain bacterially derived or fungally derived phosphatase activity. Normally, such diseases are diagnosed by culture of the infecting organism or by detecting specific antigens, antibodies or the nucleic acid by PCR. However, when the numbers of the infecting organisms are small and the host immunity is compromised (after chemotherapy or in AIDS, for example) it may be very difficult to detect some pathogenic organisms. Infections with aspergillosis is one example where the diagnosis may be difficult. In these cases it may be beneficial to look for the phosphatase associated with the pathogenic organism as this approach is very sensitive; each single organism in the infection having many molecules of phosphatase. It may be appropriate to use an antibody that is specific for the phosphatase associated with the pathogen to first capture that phosphatase before testing in order to remove any host phosphatase (eg anti-phoA has been used to immunocapture the alkaline phosphatase associated with Mycobacterium smegmatis; Kriakov et al., (2003) Journal of Bacteriology, 185:983-4991). One approach is to capture the phosphatase associated with the pathogen by using beads coated with the appropriate antibody. After capture and washing of the beads any captured phosphatase may be detected by the method described in this application. It has been observed that many alkaline phosphatases; those from bacteria for example have a very broad substrate specificity which is likely to include dsDNA labelled with end-terminal phosphate (Moura et al., Microbiology. (2001) 147:1525-33).

Another application lies in the detection of non-infectious disease. For example, the prostate is a male sex gland which produces fluid that forms part of semen. Cancer of the prostate is one of the most common types of cancer in adult males. Several tests already exist to detect Prostate cancer. Digital rectal examination may be employed to check the surface of the prostate gland. Healthy prostate tissue is typically soft, wheras malignant tissue is firm and is often assymetrical or “stony”. Transrectal ultrasounds are also used to measure the size of the prostate and visually identify tumours. Blood tests may also be used in order to check prostate specific antigen (PSA) and prostatic acid phosphatase (PAP) levels. Such tests may confirm a diagnosis made by the examinations mentioned above. PSA is produced by prostate capsule cells and periurethral glands. A highly elevated level of PSA can indicate the presence of prostate cancer. However, the PSA test can produce false positive results in the case of elevated PSA but no cancer, and also false negatives, where PSA levels are not elevated but cancer is present. Because of this, if PSA levels are high a biopsy will usually be carried out by way of confirmation. PAP is an enzyme produced by prostate tissue. The level of PAP increases as prostate disease progresses. One method used in PAP detection is Hillmans method (azo coupling of released naptha-1-ol with a diazonium compound). Lorentz (3) discusses a method that allows continuous monitoring of PAP using self-indicating substrates, the preferred substrate being 2-chloro-4-nitrophenyl phosphate (CNP—P).

Alkaline phosphatase is an important enzyme mainly derived from the liver and bones. It is found in lower amounts in the intestines, placenta, kidneys and leukocytes. Serum alkaline phosphatase has also been shown to be present at elevated levels in patients suffering from certain disease conditions. Maldonado et. al (4) have showed that serum alkaline phosphatase levels are markedly elevated in patients with sepsis, AIDS and malignancies. Wiwanitkit (5) found high serum alkaline phosphatase levels in patients with obstructive biliary diseases, infiltrative liver diseases, sepsis and cholangiocarcinoma. If serum alkaline phosphatase levels can be readily and sensitively detected this may provide a diagnostic test for a range of conditions.

### DETAILED DESCRIPTION OF THE INVENTION

As aforementioned, the present invention seeks to provide improved methods for detecting an enzyme in a sample which is capable of modifying a nucleic acid molecule by detecting the change in the nucleic acid molecule caused by the enzyme.

Such methods may be employed in a number of settings where a sensitive method of detection of an enzyme activity is required. For example, the methods of the invention may be used to enhance the sensitivity of immunological detection of an analyte and in order to provide more sensitive diagnostic methods for diagnosing specific disease conditions.

Therefore, in a first aspect of the invention there is provided a method of detecting an enzyme in a sample wherein the enzyme is capable of adding or removing a chemical moiety to or from a nucleic acid molecule, which thereby confers altered sensitivity of the nucleic acid molecule in a subsequent process, the method comprising:

allowing the sample to be tested for the presence of the enzyme to interact with the nucleic acid molecule; and

testing for interaction of the enzyme with the nucleic acid molecule by detecting the altered sensitivity of the nucleic acid molecule caused by the enzyme.

In a most preferred embodiment the enzyme will be one which may remove terminal phosphate groups from a nucleic acid molecule. Preferably said enzyme will be a phosphatase which may remove the 5′ terminal phosphate group from a nucleic acid molecule. Many phosphatases are well known in the art that may be used in accordance with the invention. The most commonly known phosphatase which has this activity is alkaline phosphatase. Alkaline phosphatase removes 5′ phosphate groups from DNA and RNA. It may also remove phosphates from nucleotides and proteins. These enzymes are most active at alkaline pH. Three major types are commonly employed in bioassays, and which may be used in the methods of the invention, although the invention is not limited to use of these specific types. Bacterial alkaline phosphatase (BAP) is a highly active enzyme. Calf intestinal alkaline phosphatase (CIP) is purified from bovine intestine, and may be inactivated using protease digestion or heat, for example. Shrimp alkaline phosphatase is derived from a cold-water shrimp and may be inactivated using heat treatment fairly readily. Further alkaline phosphatase isozymes which may be incorporated into the methods of the invention include, but are not limited to, serum, liver and bone isozymes, and those found in lower amounts in the intestines, placenta, kidneys and leukocytes.

In a further embodiment of the invention the activity of the enzyme that may remove 5′ terminal phosphates from a nucleic acid molecule, which is preferably a phosphatase and most preferably an alkaline phosphatase, will protect the nucleic acid molecule from nuclease digestion. Thus, in this specific embodiment the enzyme is capable of causing removal of a phosphate group from the 5′ end of the nucleic acid molecule, which is detectable using a suitable exonuclease, because removal of the phosphate group from the 5′ end of the nucleic acid molecule will prevent the exonuclease from acting on that molecule.

The action of a phosphatase may thus protect the nucleic acid molecule from digestion by nuclease enzymes. Exonuclease enzymes remove individual nucleotides in a processive manner from the ends of a nucleic acid molecule. Lambda exonuclease is a highly processive 5′ to 3′ exonuclease that selectively digests phosphorylated strands of double stranded DNA (dsDNA). The most preferred substrate for lambda exonuclease is blunt ended 5′ phosphorylated dsDNA. If the DNA is single stranded (ss) and/or non-phosphorylated lambda exonuclease has greatly reduced activity.

Lambda exonuclease is useful in the methods of the present invention. However, the present invention is not limited to use of lambda exonuclease. Any exonuclease which may selectively degrade phosphorylated nucleic acid molecules may be useful in the present invention. If the nucleic acid molecule employed is double stranded and blunt ended and is phosphorylated at the 5′ end lambda exonuclease will be able to rapidly digest the molecule. This digestion occurs in the absence of a suitable phosphatase in the sample being tested, such as alkaline phosphatase, which may catalyse the removal of the 5′ phosphate from the end of the nucleic acid molecule. If alkaline phosphatase activity is present in the sample the 5′ phosphate of the nucleic acid molecule is removed due to the activity of the alkaline phosphatase, thus protecting it from digestion by the lambda exonuclease. Undigested nucleic acid molecules may subsequently be detected to measure the presence of the phosphatase activity.

According to this method the preferred exonuclease is lambda exonuclease, which will digest the nucleic acid molecule if the terminal 5′ phosphate remains attached to the nucleic acid molecule.

The double stranded nucleic acid molecule utilised in various embodiments of the method of the invention may be phosphorylated at a single 5′ end or at both 5′ ends.

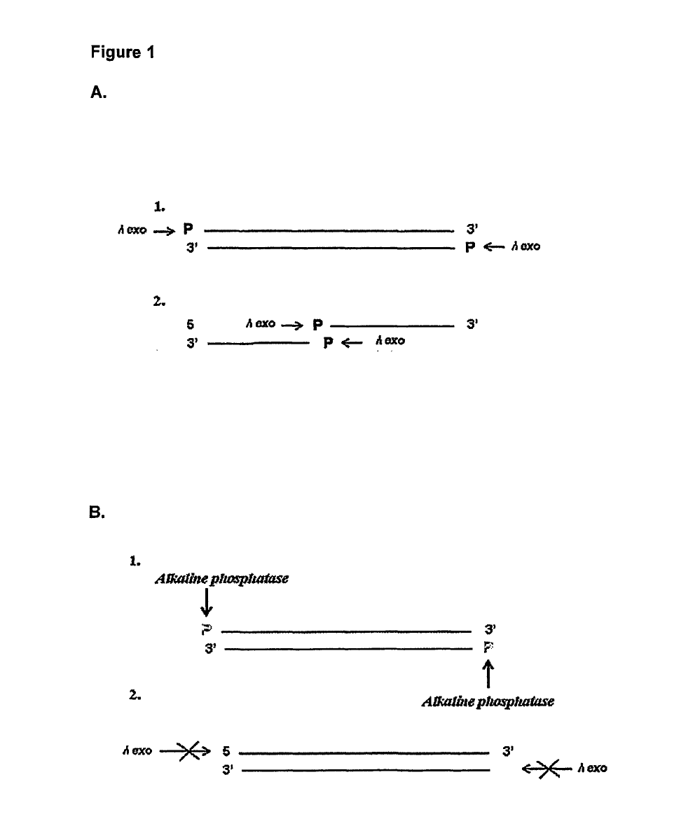

The method of the invention may need to be altered slightly depending on which type of nucleic acid molecule is being used. In the case of phosphorylation at both 5′ ends of the ds nucleic acid molecule, a detectable change in the nucleic acid will occur if a phosphatase enzyme removed either both 5′ phosphates or only a single 5′ phosphate. The probability of alkaline phosphatase catalyzing removal of both 5′ phosphates from a single (double stranded) nucleic acid molecule is reduced in comparison to removing a single 5′ phosphate, especially if the nucleic acid molecule is present in high concentration in the reaction mixture. Therefore, many of the nucleic acid molecules in the sample may still have a 5′ phosphate attached, even in the presence of phosphatase activity. This will render one of the strands (or possibly neither of them) following exposure to alkaline phosphatase in the sample, susceptible to degradation by a 5′ to 3′ exonuclease, such as lambda exonuclease. Provided at least one of the strands is protected from 5′ to 31 exonuclease activity by virtue of the dephosphorylation activity of the phosphatase, it will be possible to detect the nucleic acid strand, preferably using a nucleic acid amplification technique. For example using PCR, one of the two primers required for amplification may bind to the ssDNA (if the nucleic acid molecule used in the method is dsDNA) and this will amplify a second DNA strand to which the second primer may subsequently bind, thus allowing further amplification as more cycles of PCR are carried out. This example is described in FIG. 1 and Example 1.

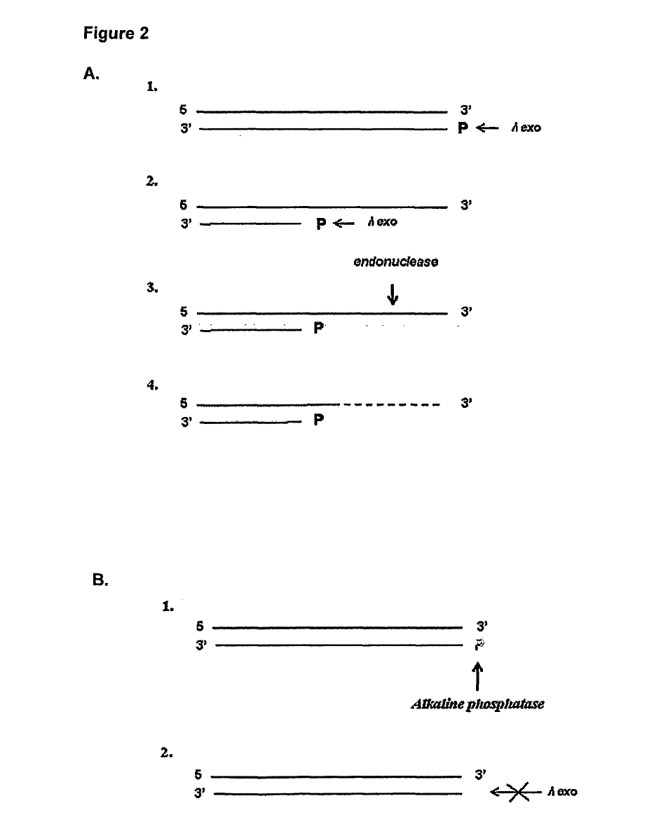

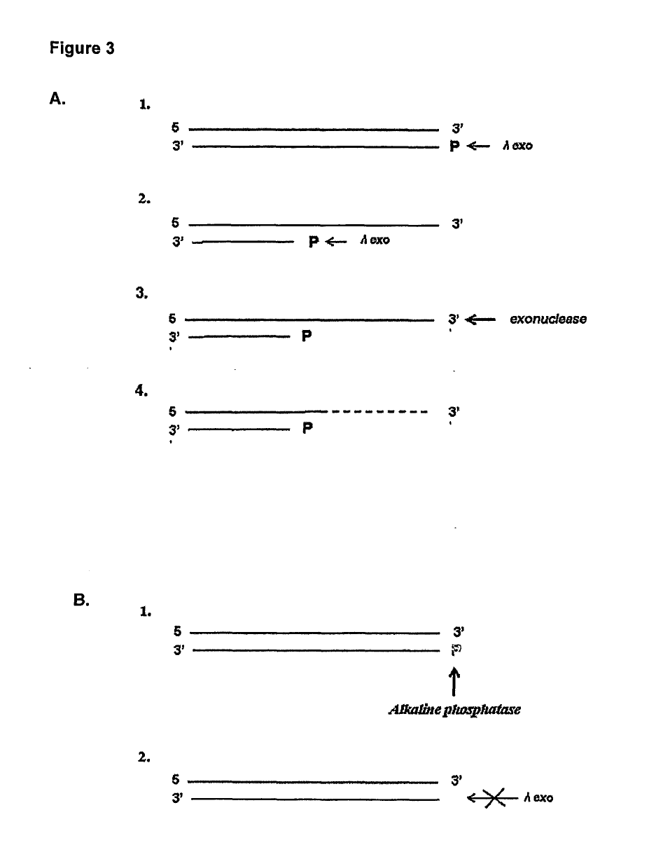

In this example lack of specificity may occur where the signal is generated even in the absence of the phosphatase. For example, if only a single 5′ end of the ds nucleic acid molecule is phosphorylated use of a 5′ to 3′ exonuclease will not be sufficient in isolation to distinguish between the presence or absence of a phosphatase because one strand will automatically be protected from 5′-3′ specific exonuclease digestion due to the lack of a phosphate group at the 5′ end and will, therefore, be available for detection, most preferably by amplification, giving a positive result even if no phosphatase activity is present. In addition, because the lambda exonuclease is specific for double stranded DNA there will be much degraded DNA left in the form of long single stranded lengths. In the subsequent PCR detection these strands may associate and be amplified and generate a non-specific signal. In this case an endonuclease may also be included in the methods of the invention in order to increase specificity. Endonucleases may hydrolyse interior bonds within a nucleic acid chain. Certain endonucleases act specifically on DNA (deoxyribonucleases) whilst others are specific for RNA (ribonucleases) (see FIG. 2). Alternatively, a complementary exonuclease such as exonuclease 1 that is specific for the 3′ end of single stranded DNA may be used to reduce the chances of single strand association, see FIG. 3 and the examples.

A complementary exonuclease is defined as one which will allow digestion of the nucleic acid molecule when used in conjunction with another exonuclease and in the absence of a phosphatase enzyme in the sample under test.

Thus, in a preferred embodiment the method of the invention further includes use of an endonuclease or a complementary exonuclease. A particularly suited endonuclease for this particular method of the invention is mung bean endonuclease, which is a ss specific endonuclease. In a most preferred embodiment the complementary exonuclease is exonuclease I, which is a single stranded (ss) nucleic acid specific exonuclease that is well known in the art

However, the invention is not limited to use of mung bean endonuclease or exonuclease I. Any endonuclease or complementary exonuclease which is specific for single stranded nucleic acid molecules may be used in this aspect of the present invention. Further examples of single strand specific endonucleases include Aspergillus nuclease S₁ (Vogt, 1973) (6) and XPF (see http://bbrp.llnl.gov/bbrp/html/thelen.abst.html). Mung bean endonuclease acts efficiently on a single stranded DNA or RNA substrate. However, mung bean endonuclease may digest dsDNA or dsRNA if present at a sufficiently high concentration.

Preferably the complementary exonuclease, most preferably exonuclease I or the endonuclease, most preferably mung bean endonuclease, is present in a sufficiently low concentration such that no, or insignificant, digestion of dsDNA or dsRNA may occur.

The nucleases for use with the invention will most preferably be added to the reaction mixture at the same time and in the same reaction mixture as the nucleic acid molecules. Here there is competition between phosphatase activity and nuclease activity. Phosphatase activity will protect the nucleic acid molecules from digestion by the exonuclease included in the reaction mixture, and thus also the single strand specific endonuclease in the case of a reaction where only a single end of the double stranded nucleic acid molecule is phosphorylated. Provided at least some phosphatase activity may take place before all nucleic acid is digested by the nuclease enzymes, this will allow detection of the protected dephosphorylated nucleic acid molecules. Therefore, most preferably, the phosphatase activity will be more efficient than nuclease activity. Suitable reaction conditions, which may favour phosphatase activity, may be incorporated in the method in order to achieve this.

Alternatively, in another embodiment, it may be possible to add the nuclease enzymes after a suitable amount of time, in a separate reagent addition step, in order to allow any phosphatase present in the test sample to have catalysed removal of terminal phosphates from the nucleic acid molecules in the test sample. A suitable amount of time is defined as one which will allow removal of a sufficient number of phosphates present on nucleic acid molecules to enable the nucleic acid molecule lacking the phosphate group to be detected and distinguished from those nucleic acid molecules which still have a terminal phosphate group attached. For any given assay system the optimal time is determined empirically, by routine experimentation. Preferably substantially all of the nucleic acid molecules are dephosphorylated before the nuclease(s) are added. Such method may increase sensitivity of the subsequent detection because more nucleic acid molecules will have had time to be dephosphorylated by phosphatase activity before the nucleases have had an opportunity to digest them and thus more nucleic acid may be detected for each phosphatase molecule present.

In a further embodiment the nucleases may be included in the initial test sample together with the nucleic acid molecules, however they may be specifically inhibited initially in order to allow the phosphatase, if present, to remove a phosphate moiety from the nucleic acid molecules. Following a suitable period to allow phosphatase activity to remove the terminal phosphates from the ds nucleic acid molecule the nucleases may be activated by removing the inhibitory conditions. For example mung bean endonuclease may be inhibited using high salt concentrations, and also requires zinc in order to be highly active. Thus by making the initial sample conditions such that there is an absence of zinc, this will allow inhibition of mung bean endonuclease activity. Mung bean endonuclease activity may be easily restored simply by adding zinc to the test sample. Such specific inhibitory conditions depend on both the phosphatase and the nucleases being employed in the method. The suitable conditions will be well known to one of skill in the art and are listed with commercially available enzymes and thus may be readily incorporated into the methods of the present invention.

Preferred Nucleic Acid Molecules

In a most preferred embodiment the nucleic acid molecule will comprise dsDNA. In further embodiments the dsDNA will be blunt ended and will be phosphorylated at either one or both 5′ ends.

In one embodiment the dsDNA molecule is produced using amplification techniques such as PCR. In this embodiment the PCR is most preferably performed using two primers that have 5′ phosphate groups. In order to ensure that the PCR product is phosphorylated it is also preferred, as an additional step, to treat the dsDNA with a kinase such as polynucleotide kinase prior to use.

In a still further embodiment the dsDNA molecule will be produced from a plasmid. If a plasmid is cut with a restriction enzyme that leaves blunt ends, linear blunt ended nucleic acid molecules will be produced having 5′ phosphate moieties at both ends. Such dsDNA molecules have advantageous characteristics for the methods of the invention. For example, if the plasmid is cut twice in defined locations two nucleic acid products are available for subsequent detection when testing for the presence of a particular enzyme activity in the sample.

In a preferred embodiment the nucleic acid molecule for use in the method of the invention will comprise commonly used vectors in molecular biology such as plasmid pUC derivatives or pBR322 or PCR derived fragments of these vectors. Any length of nucleic acid molecule may be used in the methods of the invention, provided that the addition or removal of a chemical moiety to or from the nucleic acid molecule caused by the enzyme in the sample confers altered sensitivity on the nucleic acid molecule in a subsequent process which is capable of being detected.

Preferred Detection Techniques

In order to make the technique maximally sensitive the altered sensitivity of the nucleic acid molecule in a subsequent process as caused by the addition or removal of a chemical moiety to or from the nucleic acid molecule may be detected with the use of nucleic acid amplification techniques. Such amplification techniques are well known in the art, and include methods such as PCR, NASBA (Compton, 1991), 3SR (Fahy et al., 1991), Rolling circle replication and Transcription Mediated Amplification (TMA). Amplification is achieved with the use of amplification primers specific for the sequence of the nucleic acid which is to be detected. In order to provide specificity for the nucleic acid molecules primer binding sites corresponding to a suitable region of the sequence may be selected. The skilled reader will appreciate that the nucleic acid molecules may also include sequences other than primer binding sites which are required for detection of the change in the nucleic acid molecule caused by the enzyme in the sample, for example RNA Polymerase binding sites or promoter sequences may be required for isothermal amplification technologies, such as NASBA, 3SR and TMA.

TMA (Gen-probe Inc.) is an RNA transcription amplification system using two enzymes to drive the reaction, namely RNA polymerase and reverse transcriptase. The TMA reaction is isothermal and may amplify either DNA or RNA to produce RNA amplified end products. TMA may be combined with Gen-probe's Hybridization Protection Assay (HPA) detection technique to allow detection of products in a single tube. Such single tube detection is a preferred method for carrying out the invention. This list is not intended to be exhaustive, any nucleic acid amplification technique may be used provided the appropriate nucleic acid product is specifically amplified.

Thus, in a preferred aspect of the invention the method of the invention is carried out using nucleic acid amplification techniques in order to detect the altered sensitivity of the nucleic acid molecule caused by the addition or removal of a chemical moiety. In a preferred embodiment the technique used is selected from PCR, NASBA, 3SR and TMA.

In embodiments involving the use of nucleases, the amplification method chosen will determine whether the nucleases utilised in the methods of the invention will need to be inactivated before or during the amplification step. If nuclease activity is present during amplification the products of the amplification reaction are susceptible to degradation by the nucleases present in the sample. Thus, if PCR is used to detect the altered sensitivity of the nucleic acid molecule caused by the enzyme being detected, no inactivation step is necessary because the PCR procedure begins with a heating step which will destroy any nuclease activity present. However, if an isothermal technique is utilised such as 3SR, NASBA or TMA the nuclease(s) may need to be inactivated, or removed, for example by using a suitable washing step, before the amplification takes place in order to prevent aberrant degradation of amplification products.

Detection of the amplification products may be by routine methods, such as, for example, gel electrophoresis.

A number of techniques for real-time detection of the products of an amplification reaction are known in the art. Many of these produce a fluorescent read-out that may be continuously monitored; specific examples being molecular beacons and fluorescent resonance energy transfer probes. Real-time techniques are advantageous because they keep the reaction in a “single tube”. This means there is no need for downstream analysis in order to obtain results, leading to more rapidly obtained results. Furthermore keeping the reaction in a “single tube” environment reduces the risk of cross contamination and allows a quantitative output from the methods of the invention. This may be particularly important in the diagnostic setting outlined below. Real-time quantitation of PCR reactions may be accomplished using the TaqMan® system (Applied Biosystems), see Holland et al; Detection of specific polymerase chain reaction product by utilising the 5′-3′ exonuclease activity of Thermus aquaticus DNA polymerase; Proc. Natl. Acad. Sci. USA 88, 7276-7280 (1991) (7), Gelmini et al. Quantitative polymerase chain reaction-based homogeneous assay with flurogenic probes to measure C-Erb-2 oncogene amplification. Clin. Chem. 43, 752-758 (1997)(8) and Livak et al. Towards fully automated genome wide polymorphism screening. Nat. Genet. 9, 341-342 (19995) (9) (incorporated herein by reference). Taqman® probes are widely commercially available, and the Taqman® system (Applied Biosystems) is well known in the art. Taqman® probes anneal between the upstream and downstream primer in a PCR reaction. They contain a 5′-fluorophore and a 3′-quencher. During amplification the 5′-3′ exonuclease activity of the Taq polymerase cleaves the fluorophore off the probe. Since the fluorophore is no longer in close proximity to the quencher, the fluorophore will be allowed to fluoresce. The resulting fluorescence may be measured, and is in direct proportion to the amount of target sequence that is being amplified.

In the Molecular Beacon system, see Tyagi & Kramer. Molecular beacons—probes that fluoresce upon hybridization. Nat. Biotechnol. 14, 303-308 (1996) (10) and Tyagi et al. Multicolor molecular beacons for allele discrimination. Nat. Biotechnol. 16, 49-53 (1998) (11)(incorporated herein by reference), the beacons are hairpin-shaped probes with an internally quenched fluorophore whose fluorescence is restored when bound to its target. The loop portion acts as the probe while the stem is formed by complimentary “arm” sequences at the ends of the beacon. A fluorophore and quenching moiety are attached at opposite ends, the stem keeping each of the moieties in close proximity, causing the fluorophore to be quenched by energy transfer. When the beacon detects its target, it undergoes a conformational change forcing the stem apart, thus separating the fluorophore and quencher. This causes the energy transfer to be disrupted to restore fluorescence.

Any suitable fluorophore is included within the scope of the invention. Fluorophores that may possibly be used in the method of the invention include, by way of example, FAM, HEX™, NED™, ROX™, Texas Red™ etc. Quenchers, for example Dabcyl and TAMRA are well known quencher molecules that may be used in the method of the invention. However, the invention is not limited to these specific examples.

A further real-time fluorescence based system which may be incorporated in the methods of the invention is Zeneca's Scorpion system, see Detection of PCR products using self-probing amplicons and fluorescence by Whitcombe et al. Nature Biotechnology 17, 804-807 (1 Aug. 1999) (12). This reference is incorporated into the application in its entirety. The method is based on a primer with a tail attached to its 5′ end by a linker that prevents copying of the 5′ extension. The probe element is designed so that it hybridizes to its target only when the target site has been incorporated into the same molecule by extension of the tailed primer. This method produces a rapid and reliable signal, because probe-target binding is kinetically favoured over intrastrand secondary structures.

Thus, in a further aspect of the invention the products of nucleic acid amplification are detected using real-time techniques. In one specific embodiment of the invention the real-time technique consists of using any one of the Taqman® system, the Molecular beacons system or the Scorpion probe system.

In a most preferred embodiment the reaction mixture will contain all of; the sample under test, the nucleic acid molecules, the required nucleases and buffers and all reagents, buffers and enzymes required for amplification in addition to the reagents required to allow real time detection of amplification products. Thus the entire detection method for the enzyme of interest, most preferably a phosphatase, will occur in a single reaction, with a quantitative output, and without the need for any intermediate washing steps. Use of a “single tube” reaction is advantageous because there is no need for downstream analysis in order to obtain results, leading to more rapidly obtained results. Furthermore keeping the reaction in a “single tube” environment reduces the risk of cross contamination and allows a quantitative output from the methods of the invention. Also, single tube reactions are more amenable to automation, for example in a high throughput context.

Alternatively the method of the invention may be carried out in step-wise fashion. Thus, the nucleic acid molecules may be added first to the sample under test, allowing any enzyme present in the sample to change the nucleic acid molecule. Following this, in one embodiment, the nuclease enzymes may be added to digest unchanged nucleic acid molecules. This may involve changing the reaction conditions in the sample. The nuclease may then, in a further embodiment, be inactivated before adding reagents necessary for detection, which will most preferably be by amplification. Depending on whether an isothermal amplification technique is used this will determine whether the nucleases will need to be inactivated before carrying out the detection step. If real time detection is being utilised the required reagents are added together with the reagents required for the amplification stage.

Primers specific for the nucleic acid molecule to be amplified are utilised in the methods and kits of the invention. Any primer that may direct sequence specific amplification with minimum background, non-specific amplification, may be utilised. Primers may comprise DNA or RNA and synthetic equivalents depending upon the amplification technique being utilised. For example for standard PCR a short single stranded DNA primer pair tends to be used, with both primers bordering a region of interest to be amplified. The types of primers that may be used in nucleic acid amplification technology such as PCR, 3SR, NASBA and TMA are well known in the art.

Suitable probes for use in the real-time methods may also be designed, in order that they may be used in conjunction with the nucleic acid molecules in the methods of the invention. Thus, for example, when using the Taqman® technique, the probes may need to be of sequence such that they can bind between primer binding sites on the nucleic acid molecule which is modified by an enzyme to give the change that is subsequently detected in real-time. Similarly molecular beacon probes may be designed that bind to a relevant portion of the nucleic acid sequence incorporated into the methods and kits of the invention. If using the Scorpion probe technique for real time detection the probe will need to be designed such that it hybridizes to its target only when the target site has been incorporated into the same molecule by extension of the tailed primer. Therefore, the invention further provides for inclusion of probes suitable for use in real-time detection methods in the present invention.

Alternative techniques may be used to detect the addition of a chemical moiety to, or removal of a chemical moiety from, the nucleic acid molecule. Such a detection step may, in one embodiment, be sensitive enough to detect the removal of a phosphate group from the terminal end of the nucleic acid molecule without the need to include nucleases, as described above, to digest any nucleic acid molecules from which the phosphate moiety has not been removed. However, in this case it is necessary to ensure that substantially all nucleic acid molecules are initially phosphorylated. If they are not, an apparently positive result may be achieved even in the absence of phosphatase activity in the sample.

Alternatively the nucleases referred to above may also be included in the reaction mixture, in a further embodiment of the invention, so that any nucleic acid molecules which have not had the phosphate moiety removed are degraded and thus will not be detected by the alternative techniques described below. Examples of alternative detection techniques include mass spectrometry, including matrix assisted laser desorption (MALDI) mass spectrometry and MALDI-Time of Flight (MALDI-TOF) mass spectrometry, chromatography and use of microarray technology (Motorola, Nanogen). Mass spectrometry will allow the expected molecular weight of the nucleic acid molecules to be accurately measured. MALDI-TOF relies upon a high voltage potential which rapidly extracts ions and accelerates them down a flight tube. A detector at the end of the flight tube is used to determine the time elapsed from the initial laser pulse to detection of the ions. The flight time is proportional to the mass of the ion. Thus, even in the absence of nucleases in certain embodiments of the method of the invention the difference in the mass of the ds nucleic acid molecules, depending on whether a phosphate group is attached at the 5′ terminal end, may still be detected to distinguish between those nucleic acid molecules which retain the phosphate moiety and those where the phosphate moiety has been removed. Obviously, if the nucleic acid molecules which retain the phosphate moiety are digested using suitable nucleases, the difference in mass is more readily detectable between nucleic acid molecules which retain the phosphate moiety and those that do not.

Similarly, by using a microarray with suitable tags attached to the solid support, the nucleic acid molecules for which a chemical moiety has been added or removed due to the enzyme activity in the sample may be identified in a downstream process. Again this technique may be able to distinguish phosphorylated from unphosphorylated nucleic acid molecules, or alternatively, nuclease digestion may be used to remove those nucleic acid molecules which retain the chemical moiety.

These alternative techniques may preferably be used in conjunction with nucleic acid amplification techniques in order to characterise the amplification products. This will help to remove false positive results, where an amplified product had been produced which is not the expected product. Thus the advantages of an amplification step to increase sensitivity is combined with a step to accurately characterise the amplification products thus making the methods of the invention even more accurate.

Immunoassays

In a particular embodiment the method of the invention can be used to enhance the sensitivity of any assay system which is based upon detection of phosphatase activity. In a most preferred embodiment the method of the invention may advantageously be used to enhance the sensitivity of an immunoassay, such as a Western blot, dot blot, ELISA, immunoprecipitation or immunodiffusion for example. However, the invention is not intended to be restricted to only these examples.

In many immunoassays a primary antibody will be used which is specific for the antigen to be detected. In order to detect binding of the first, typically unlabelled, antibody to the antigen, following a washing step to remove unbound antigen, a secondary antibody will be added which cross reacts with the primary antibody. This secondary antibody is often conjugated to an enzyme such as horseradish peroxidase or alkaline phosphatase. Alternatively, for solution-based assays such as ELISAs, immunoprecipitation and immunodiffusion the secondary antibody recognises a second site on the antigen. Again, the secondary antibody is often conjugated to an enzyme such as HRP or AP. Such enzymes can react with a substrate chromogen to give a coloured product in the presence of an antigen. For example, a commonly used substrate chromogen used with alkaline phosphatase is 5-bromo, 4-chloro, 3-indolylphosphate (BCIP). An additive such as iodoblue tetrazolium (INT) may also be used to enhance the final colour of the precipitate at the reaction sites, that is where the primary and secondary antibodies have bound to the antigen (which is a yellow-brown colour for BCIP with INT). Many fluorogenic substrates for HRP are well known in the art and are commercially available. One example is Amplex Red Reagent (Molecular Probes), 10-acetyl-3,7-dihydroxyphenoxazine, which can react with H₂O₂ in a 1:1 stochiometry in the presence of HRP to produce highly fluorescent resorufin. An alternative substrate is scopoletin, where HRP catalyzes conversion of the fluorescent scopoletin to a nonfluorescent product. Such substrates are commonly included in ELISA kits to allow detection of sites where an antigen/analyte is present.

The inventors have utilised the fact that enzymes commonly used in immunoassays, such as alkaline phosphatase, can also act upon nucleic acid substrates to give a detectable change. Phosphatases, such as calf intestinal phosphatase (CIP) for example, may remove the 5′ terminal phosphate group from a double stranded DNA (dsDNA) molecule. Any phosphatase capable of such activity is included within the scope of the present invention. This activity is significantly more efficient when the DNA is blunt ended, that is where there are no single stranded (ss) overhangs. By including suitable nucleic acid molecules in the immunoassay, the presence of an analyte/antigen may be detected in a sensitive manner by monitoring the altered sensitivity of the nucleic acid molecule caused by the (antibody conjugated) enzyme which is capable of either adding or removing a chemical moiety to or from the nucleic acid molecule.

Obrecht and Dirheimer (13) have shown that HRP can catalyse formation of DNA and deoxyguanosine 3′-monophosphate (dGMP) adducts in vitro in the presence of Ochratoxin A (OTA). The reaction is less efficient in the presence of hydrogen peroxide (H₂O₂) than in the presence of cumene hydroperoxide. The peroxidase can metabolize OTA to form an activated species that can bind covalently to DNA and dGMP. Thus it may be possible to use the method of the invention by detecting for HRP's ability to form an adduct between DNA and other molecules, such as dGMP in the presence of OTA. The adduct may be detected by well known methods in the art such as chromatography, or mass spectrometry, such as MALDI or MALDI-TOF mass spectrometry, and use of microarray technology (Motorola, Nanogen) for example.

Thus in one preferred embodiment the method of the invention is carried out to detect the presence of an enzyme wherein the enzyme is one which is used for detection of an antigen/analyte in an immunoassay. Preferably, the enzyme that will be detected is attached to an antibody which is used in the detection of an antigen/analyte. The antibody may be a primary antibody or a secondary antibody.

However, the method of the invention is not limited to use for enhancing the sensitivity of immunoassays. As aforementioned Alkaline phosphatase-conjugated oligonucleotides/probes (Sigma-Genosys) may be used for routine screening applications such as Southern (DNA) and Northern (RNA) blotting, gene mapping and restriction fragment length polymorphism (RFLP) analysis. They may also be used for in situ hybridizations. The methods of the present invention may be utilised to enhance the sensitivity of such techniques. Instead of using calorimetric detection of AP activity the method of the present invention may be used in order to detect AP activity and thus probe binding. By coupling AP's ability to modify a nucleic acid molecule to an amplification step to detect the modified nucleic acid, sensitivity is increased. Care will need to be taken to ensure that the oligonucleotides conjugated to the AP molecules will not interfere with the detection of the nucleic acid molecule being modified by AP. Also the actual nucleic acid sequences being probed may need to be treated to prevent interference with the method of the invention. Nuclease digestion by lambda exonuclease may, for example, be prevented by utilising probes and sample sequences which are not phosphorylated at their 5′ ends. This may be achieved by carrying out a dephosphorylation step as a precursor to the detection step. This will necessarily occur before adding the ds nucleic acid molecules which are phosphorylated at one or both 5′ ends, otherwise the change in the nucleic acid molecule caused by phosphatase activity may not take place, and thus enzyme activity in the sample may not be sensitively detected.

Detection of Infectious Disease

As aforementioned, the method of the invention may be applied to detect free phosphatase associated with an infectious agent.

Thus, there is provided a method of detecting a phosphatase from an infectious agent in a sample wherein the phosphatase is capable of adding or removing a chemical moiety to or from a nucleic acid molecule, which thereby confers altered sensitivity of the nucleic acid molecule in a subsequent process, the method comprising:

allowing the sample to be tested for the presence of the phosphatase to interact with the nucleic acid molecule; and

testing for interaction of the phosphatase with the nucleic acid molecule by detecting the altered sensitivity of the nucleic acid molecule caused by the phosphatase, wherein detection of altered sensitivity indicates the presence of the infectious agent.

In one embodiment the infectious agent is Aspergillus or Staphyloccocus species.

The sample will generally be one taken from a subject suspected of being infected by the infectious agent. Any type of sample may be used in which the infectious agent may be present. Tissue and cell samples will generally be utilised although whole blood, serum, plasma, urine, chyle, stool, ejaculate, sputum, nipple aspirate, saliva etc. taken from a subject may also be tested in the method.

The subject is most preferably a human subject, but may include an animal subject such as a dog, cat, pig, cow or monkey for example.

The method is intended to be an in vitro method utilising an isolated sample. However, in one embodiment the method may additionally comprise the step of obtaining a suitable sample from the subject under test.

In a most preferred embodiment, and in order to ensure that phosphatase enzyme associated with an infectious agent is distinguished from host phosphatase enzyme, the method additionally comprises the substeps of:

a) capture and separation of the infectious agent-specific phosphatase via a specific antibody

a) adding to the separated phosphatase a nucleic acid molecule which comprises blunt ended dsDNA which is phosphorylated at both 5′ ends

b) incubating under conditions which permit phosphatase activity

c) adding lambda exonuclease to the sample and allowing incubation with this enzyme; and

d) detecting the altered sensitivity of the nucleic acid molecule, measured as the presence or absence of the nucleic acid molecule, wherein detection of altered sensitivity indicates the presence of the infectious agent.

Diagnostic Methods for Detection of Non-Infectious Disease

Many phosphatases are known to have disease associations. For example elevated levels of prostatic acid phosphatase (PAP) are known to be linked to prostate cancer. By utilising suitable nucleic acid molecules, capable of being dephosphorylated by PAP, a diagnostic test for prostate cancer may fall within the scope of the present invention.

Alkaline phosphatase is an important enzyme mainly derived from the liver and bones. It is found in lower amounts in the intestines, placenta, kidneys and leukocytes. Furthermore, alkaline phosphatase levels in serum have been shown to be increased in subjects suffering from a range of conditions. Maldonado et. al (3) have showed that serum alkaline phosphatase levels are markedly elevated in patients with sepsis, AIDS and malignancies. Wiwanitkit (4) found high serum alkaline phosphatase levels in patients with obstructive biliary diseases, infiltrative liver diseases, sepsis and cholangiocarcinoma. By sensitively detecting serum alkaline phosphatase using the method of the invention a diagnostic test may be envisaged for diagnosing each of these conditions, without the need for a large sample from the patient.

Thus, the invention provides a method of diagnosing prostate cancer in a mammalian subject comprising

allowing a sample obtained from the subject under test to be tested for the presence of prostatic acid phosphatase (PAP) to interact with a nucleic acid molecule; and

testing for interaction of PAP with the nucleic acid molecule by detecting the altered sensitivity in the nucleic acid molecule caused by PAP (in a downstream process), whereby the presence of prostatic acid phosphatase (PAP) in the sample is taken as an indication that the subject may have or has prostate cancer.

Similarly the invention provides a method of diagnosing a disease associated with elevated serum alkaline phosphatase levels, including any one of sepsis, AIDS, malignancies, obstructive biliary diseases, infiltrative liver diseases, sepsis and cholangiocarcinoma in a mammalian subject comprising

allowing a sample obtained from the subject to be tested for the presence of serum alkaline phosphatase to interact with a nucleic acid molecule; and

testing for interaction of serum alkaline phosphatase with the nucleic acid molecule by detecting an altered sensitivity in the nucleic acid molecule caused by serum alkaline phosphatase (in a downstream process), whereby the presence of serum alkaline phosphatase in the sample is taken as an indication that the subject may have or has the disease, which may be any one of sepsis, AIDS, malignancies, obstructive biliary diseases, infiltrative liver diseases, sepsis and cholangiocarcinoma.

In this context the “sample” will generally be a clinical sample. The sample being used will depend on the condition that is being tested for. In the case of diagnosing prostate cancer a suitable prostate sample from the patient may be required. Alternatively a blood sample may be utilised, since elevated PAP levels are found in the blood of a patient suffering from prostate cancer. Typical samples which may be used, but which are not intended to limit the invention, include whole blood, serum, plasma, urine etc. taken from a patient, most preferably a human patient.

In a most preferred embodiment the test will be an in vitro test carried out on a sample removed from a subject.

In a further embodiment the above-described diagnostic methods may additionally include the step of obtaining the sample from a subject. Methods of obtaining a suitable sample from a subject are well known in the art. Alternatively, the method may be carried out beginning with a sample that has already been isolated from the patient in a separate procedure. The diagnostic methods will most preferably be carried out on a sample from a human, but the method of the invention may have diagnostic utility for many animals.

The diagnostic methods of the invention may be used to complement any already available diagnostic techniques, potentially as a method of confirming an initial diagnosis. Alternatively, the methods may be used as a preliminary diagnosis method in their own right, since the methods will provide a quick and convenient diagnostic method. Furthermore, due to their inherent sensitivity, the diagnostic methods of the invention will require only a minimal sample, thus preventing unnecessary invasive surgery.

Kits

The invention also provides kits which may be used in order to carry out the methods of the invention. The kits may incorporate any of the preferred features mentioned in connection with the methods of the invention above.

Accordingly, in a further aspect of the invention there is provided a kit for detecting an enzyme capable of adding or removing a chemical moiety to or from a nucleic acid molecule, which thereby confers altered sensitivity of the nucleic acid molecule in a subsequent process, comprising:
a nucleic acid molecule which is capable of being acted upon by the enzyme; and
means for detecting the altered sensitivity of the nucleic acid molecule in the subsequent process.

The kit may advantageously be used to complement already available kits which are based on using the target enzyme in question. Thus, for example, a standard ELISA kit will probably contain a suitable chromogenic or chemiluminescent substrate in order to detect if the enzyme, such as horseradish peroxidase or alkaline phosphatase has, in fact, bound via an antibody to the site where an antigen/analyte is present. This step of detecting enzyme activity may be replaced by the kit of the invention, which may advantageously add an extra amplification step to sensitise the detection of an analyte/antigen still further.

In a preferred embodiment the kit may be used to enhance the sensitivity of an immunoassay which includes alkaline phosphatase as the enzyme to detect binding of the antibody to the analyte/antigen, by utilising alkaline phosphatase's ability to remove 5′ terminal phosphates from DNA and RNA molecules. Thus in a preferred embodiment a kit is provided wherein the nucleic acid molecule is dsDNA. A further preferred feature is to include in the kit nucleic acid molecules which are blunt ended. Furthermore the nucleic acid molecules will preferably be phosphorylated at one or both 5′ ends, to allow the phosphatase, if present in a sample, to act on the nucleic acid molecule by removing the 5′ terminal phosphate(s).

In a further aspect the kits of the invention may include a nucleic acid molecule which comprises a plasmid which can be cut using restriction enzymes to give blunt ended dsDNA which is phosphorylated at both 5′ ends. This will effectively allow the kit, following the treatment of the plasmid with the appropriate restriction enzymes, to comprise at least one linear dsDNA molecule, which is blunt ended and phosphorylated at both 5′ ends. Such nucleic acid molecules will prove useful in the methods of the invention. If more than one restriction enzyme site is present in the plasmid the nucleic acid may be cut into a number of separate linear dsDNA molecules, preferably blunt-ended and preferably phosphorylated at both 5′ ends.

Accordingly, the kits of the invention may further including the restriction enzymes necessary to cut the plasmid. Any suitable restriction enzyme may be used, most preferably one which gives blunt ended cuts in the nucleic acid molecule and leaves the 5′ ends of the molecule phosphorylated. Many such restriction enzymes are commercially available. In fact, most restriction enzymes cleave nucleic acid sequences to leave 5′-phosphate and 3′-hydroxyl ends (although Nci I generates 3′-phosphate and 5′-hydroxyl ends). Restriction enzymes which recognize a palindromic sequence can often cut to leave a blunt end with no protruding bases. These restriction enzymes are preferred in the kits of the invention.

As mentioned above, in the description relating to the methods of the invention, nucleases may be incorporated which will digest the nucleic acid molecules in the absence of enzyme activity which either adds or removes a chemical moiety to or from the nucleic acid molecule. In order to provide for these methods a kit is provided wherein the means for measuring the nucleic acid molecule with altered sensitivity as a result of enzyme activity includes an exonuclease and/or an endonuclease which digests the nucleic acid molecule if no enzyme activity is present to cause the addition or removal of a chemical moiety to or from the nucleic acid molecule.

In the method where alkaline phosphatase is detected and either a single end or both ends of the nucleic acid molecule are 5′ phosphorylated a 5′ to 3′ processive exonuclease is useful in the method. Thus a kit is provided wherein the exonuclease comprises a 5′-3′ processive exonuclease. In a most preferred embodiment this exonuclease comprises lambda exonuclease.

In a preferred embodiment, for use in the method where both 5′ ends of the nucleic acid molecule are phosphorylated, the kit may further include an endonuclease, preferably specific for single stranded nucleic acids, which endonuclease will most preferably comprise mung bean endonuclease.

As mentioned above the method of the invention will prove maximally sensitive when the altered sensitivity of the nucleic acid molecule caused by the addition or removal of a chemical moiety as catalysed by the modifying enzyme is detected using nucleic acid amplification techniques. As aforementioned preferred amplification techniques include PCR, Rolling circle replication, NASBA, 3SR and TMA techniques. In the case of nucleic acid amplification techniques, well known in the art, sequence specific primers are required to allow specific amplification of the product with minimal production of false positive results. To this end, the kits of the invention may preferably include sequence specific primers.

The kit may also include reagents necessary for a nucleic acid amplification step. Reagents may include, by way of example and not limitation, amplification enzymes, probes, positive control amplification templates, reaction buffers etc. For example, in the PCR method, possible reagents include a suitable polymerase such as Taq polymerase and appropriate PCR buffers, and in the TMA method the appropriate reagents include RNA polymerase and reverse transcriptase enzymes. All of these reagents are commercially available and well known in the art.

The kit may further include components required for real time detection of amplification products, such as fluorescent probes for example. As aforementioned the relevant real-time technologies, and the reagents required for such methods, are well known in the art and are commercially available. Suitable probes for use in these real-time methods may also be designed, in order that they may be used in conjunction with the nucleic acid molecules incorporated into the kits of the invention for their ability to be modified by appropriate enzyme activity. Thus, for example using the Taqman® technique, the probes may need to be of sequence such that they can bind between PCR primer sites on the nucleic acid molecule whose sensitivity in a downstream process has been modified by the activity of an enzyme which either adds or removes a chemical moiety that is subsequently detected in real-time. Similarly, molecular beacons probes may be designed that bind to a relevant portion of the nucleic acid sequence incorporated into the kits of the invention. If using the Scorpion probe technique for real time detection the probe will need to be designed such that it hybridizes to its target only when the target site has been incorporated into the same molecule by extension of the tailed primer. Suitable probes are accordingly included in a further aspect of the kits of the invention.

A kit for detection of a phosphatase associated with an infectious agent is also provided comprising:
an antibody selective for an infectious agent-specific phosphatase;
a nucleic acid molecule which is capable of being acted upon by the phosphatase associated with an infectious agent in order to cause an altered sensitivity in the nucleic acid molecule in a downstream process; and
means for detecting the altered sensitivity of the nucleic acid molecule in a downstream process.

In one embodiment the infectious agent is Aspergillus or Staphyloccocus species.

### EXPERIMENTAL SECTION

The invention will be further understood with reference to the following examples, together with the accompanying tables and figures in which:

FIG. 1—In this method the alkaline phosphatase is detected by dephosphorylation and subsequent protection from lambda exonuclease of both ends of a DNA template.

A. In the absence of alkaline phosphatase the lambda exonuclease (lambda exo) recognizes the phosphate group (P) on both strands of the double-stranded DNA and degrades these strands (see 1 and 2). In a subsequent PCR with primers that are specific for this DNA sequence, the degraded DNA sequence will not produce any PCR product.

B. In the presence of alkaline phosphatase either or both the phosphate groups on the 5′ ends of the double-stranded DNA are removed by the phosphatase (see 1, this shows a situation where both phosphate groups are removed) and the DNA is no longer recognized by the lambda exonuclease (see 2). This protects the DNA from degradation. In a subsequent PCR with primers that are specific for this DNA sequence, the undegraded DNA sequence is a template for PCR and will produce the characteristic PCR product.

FIG. 2—In this method only one end of the double-stranded DNA is susceptible to λlambda exonuclease in having a 5′ phosphate group.

A. In the absence of alkaline phosphatase the lambda exonuclease (lambda exo) recognizes the phosphate group (P) on the one strand of the double stranded DNA and degrades this strand (see 1 and 2). As the strand degrades this exposes single-stranded DNA on the opposite strand which is degraded by a single-strand specific endonuclease eg mung bean endonuclease (see 3 and 4). In a subsequent PCR with primers that are specific for this DNA sequence, the degraded DNA sequence will not produce any PCR product.

B. In the presence of alkaline phosphatase the single phosphate group on the 5′ end of the double-stranded DNA is removed by the phosphatase (see 1) and the DNA is no longer recognized by the lambda exonuclease (see 2). This protects the DNA from degradation by both the lambda exonuclease and the single-stranded mung bean nuclease. In a subsequent PCR with primers that are specific for this DNA sequence, the undegraded DNA sequence is a template for PCR and will produce the characteristic PCR product.

FIG. 3 In this method only one end of the double-stranded DNA is susceptible to lambda exonuclease in having a 5′ phosphate group.

A. In the absence of alkaline phosphatase the lambda exonuclease (lambda exo) recognizes the phosphate group (P) on the one strand of the double stranded DNA and degrades this strand (see 1 and 2). As the strand degrades this exposes single-stranded DNA on the opposite strand which is degraded by a 3′ single-strand specific exonuclease eg exonuclease 1 (see 3 and 4). In a subsequent PCR with primers that are specific for this DNA sequence, the degraded DNA sequence will not produce any PCR product.

B. In the presence of alkaline phosphatase the single phosphate group on the 5′ end of the double-stranded DNA is removed by the phosphatase (see 1) and the DNA is no longer recognized by the lambda exonuclease (see 2). This protects the DNA from degradation by both the lambda exonuclease and the single-stranded mung bean nuclease. In a subsequent PCR with primers that are specific for this DNA sequence, the undegraded DNA sequence is a template for PCR and will produce the characteristic PCR product.

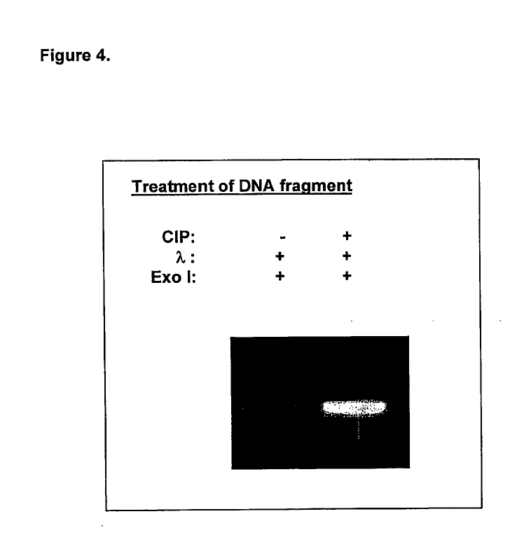

FIG. 4. Detection of alkaline phosphatase via inclusion of exonuclease I. The addition of exonuclease I is effective in reducing the background in the reaction without CIP (CIP−). In the reaction treated with CIP (CIP+) a clear signal can be seen after PCR amplification.

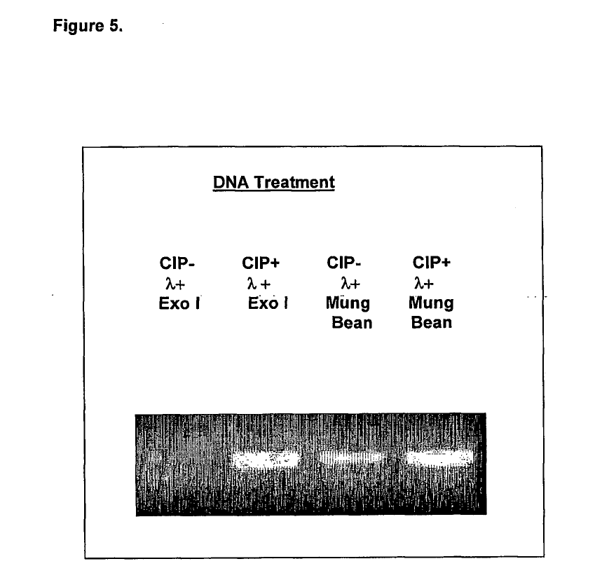

FIG. 5. Comparison of Mung Bean endonuclease and exonuclease 1 in the detection of alkaline phosphatase. Mung Bean endonuclease can be used in the assay but it is not as effective as exonuclease I in reducing the background signal in reactions without CIP.

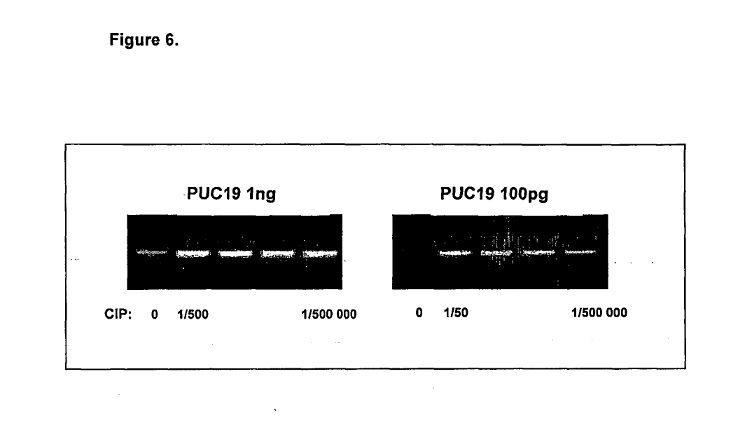

FIG. 6. Sensitivity of detection of alkaline phosphatase via inclusion of exonuclease I. The addition of exonuclease I is effective in reducing the background in the reaction without CIP (CIP−). In the reaction treated with CIP (CIP+) a clear signal can be seen after PCR amplification.

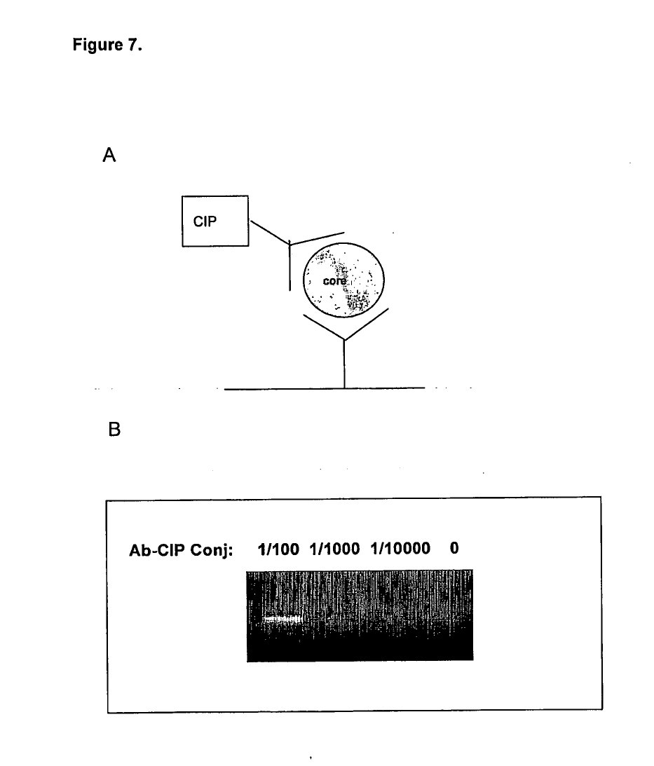

FIG. 7. Detection of alkaline phosphatase via inclusion of exonuclease I performed with an immobilized alkaline phosphatase-antibody conjugate.

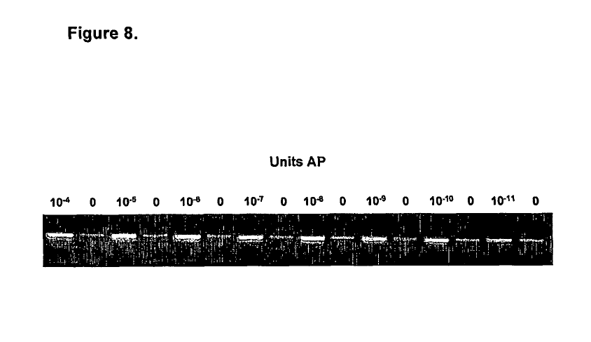

FIG. 8 shows the PCR products from the ‘detection’ step, separated by agarose gel electrophoresis and stained with ethidium bromide. Visual inspection indicates a detection limit of less than 10⁻¹¹ Units of alkaline phosphatase, which is approximately equivalent to 10×10⁻¹⁸ g, or as few as 60 molecules of alkaline phosphatase (AP) in our assay.

Method 1

Background

Alkaline phosphatase can be detected using plasmid DNA as a substrate. The plasmid was cut with a restriction enzyme to yield blunt-ended 5′ phosphorylated double-stranded DNA. This DNA can be degraded by lambda exonuclease which is specific for double stranded 5′ phosphorylated DNA. Removal of the phosphate groups by alkaline phosphatase renders the DNA resistant to the exonuclease digestion. This resistant DNA can be detected by nucleic acid amplification methods; in this example by the polymerase chain reaction using primers specific for the plasmid DNA.

Method

1. pUC19 DNA was digested to completion with the restriction enzyme PvuII (New England Biolabs, NEB).
2. Serial 10-fold dilutions of the antibody alkaline phosphatase conjugate (anti-mouse IgG alkaline phosphatase from Sigma Aldrich Catalogue number A3563) were prepared in 10 μl reaction buffer containing 0.3×NEB buffer 3 (supplied as a 10× stock) and 1 ng cut plasmid DNA.
3. The reaction was incubated for 1 hour at 37° C.
4. After dephosphorylation, 10 μof 1× lambda exonuclease buffer containing 5 units of lambda exonuclease (NEB) were added to each reaction and incubated at 37° C. for 30 mins.
5. The reactions were then heated at 95° C. for 5 mins and 2 μl of each reaction analysed by PCR using primers specific for the plasmid DNA and 20 cycles of PCR.
6. After PCR, the PCR products were analyzed by agarose gel electrophoresis.
   Results

Control reactions without any alkaline phosphatase produced no PCR products; the phosphorylated plasmid DNA was completely digested by the nuclease and could not be amplified. Pre-treatment of the plasmid DNA with alkaline phosphatase removed the phosphate groups from the DNA and rendered it resistant to nuclease digestion. This intact DNA could subsequently be amplified by PCR. Using this approach the plasmid DNA in the serial dilutions containing an amount of alkaline phosphatase equal to or greater than 1 picogram could be amplified by PCR and detected on the agarose gel.

2. Discussion

This demonstrates that the activity of alkaline phosphatase present as an antibody conjugate can be translated through action on a DNA template to a signal by PCR. In addition, the method is a highly sensitive method for the detection of alkaline phosphatase.

Method 2

Detection of Alkaline Phosphatase Via Inclusion of Exonuclease I

Background

Exonuclease I catalyzes the removal of nucleotides from single-stranded DNA in the 3′ to 5′ direction. When phosphorylated DNA (non-protected) is digested with lambda exonuclease intermediate single-stranded structures will be formed (FIG. 3, A2). During the polymerase detection reaction these can provide a template for the PCR primers resulting in a product. This would provide a false positive result. The addition of exonuclease I degrades these structures completely down to nucleotides thereby eliminating or reducing background.

Method

1. Approximately 10 ng of a purified blunt-ended cut fragment from pUC19 in 10 μl of 1× Calf Intestinal Alkaline phosphatase buffer (NEB), was incubated with 1 μl of a 1/500 dilution of Calf Intestinal Alkaline phosphatase (CIP) (NEB) at 37° C. for 1 hr (see CIP+ in the figure below). Another reaction was performed without any CIP (see CIP− in the figure below).
2. 1 μl lambda exonuclease (NEB) at a 1/10 dilution was then added to each of the above reactions and the reaction volume, made up to 20 μl in 1× lambda exonuclease buffer (NEB) and incubated at 37° C. for 30 min.
3. The enzyme was inactivated by incubation at 75° C. for 10 min.
4. 1 μl exonuclease I (NEB) diluted 1/10 was then added to each reaction and incubated at 37° C. for 30 min.
5. The enzyme was inactivated by incubation at 80° C. for 20 min.
6. 5 μl of each reaction was removed and used as template in a PCR reaction with internal primers for the fragment that generate a 250 bp fragment.
7. Products were analyzed by gel electrophoresis and visualized by ethidium bromide staining (see results below).
   Results

Results are shown in FIG. 4

Discussion

The addition of exonuclease I is effective in reducing the background in the reaction without CIP (CIP−, above). In the reaction treated with CIP (CIP+, above) a clear signal can be seen after PCR amplification.

Comparison of Mung Bean endonuclease and exonuclease 1 in the detection of alkaline phosphatase

Background

Mung Bean Nuclease is an endonuclease which catalyzes the removal of single-stranded DNA extensions (3′ and 5′) to leave blunt ends. This was added to the reaction after lambda digestion to test its efficiency at degrading any remaining single-stranded structures and hence reduce or eliminate background. A comparison with exonuclease I was performed to compare the two enzymes in the assay. The preferable reaction would result in full degradation of DNA without CIP.

Method

1. Approximately 10 ng of a purified blunt-ended cut fragment from pUC19 in 10 μl of 1× Calf Intestinal Alkaline phosphatase buffer (NEB), was incubated with 1 μl of a 1/500 dilution of Calf Intestinal Alkaline phosphatase (CIP) (NEB) at 37° C. for 1 hr (see CIP+ in the figure below). Another reaction was performed without any CIP (see CIP− in the figure below).
2. 1 μl lambda exonuclease (NEB) at a 1/10 dilution was then added to each of the above reactions and the reaction volume, made up to 20 μl in 1× lambda exonuclease buffer (NEB) and incubated at 37° C. for 30 min.
3. The enzyme was inactivated by incubation at 75° C. for 10 min.
4. 1 μl of either Mung Bean Nuclease (NEB) diluted 1/10 or Exonuclease I (NEB) diluted 1/10 was then added to each reaction and incubated at 37° C. for 30 min.
5. The enzymes were inactivated by incubation at 80° C. for 20 min.
6. 5 μl of each reaction was removed and used as template in a PCR reaction with internal primers for the fragment that generate a 250 bp fragment.
7. Products were analyzed by gel electrophoresis and visualized by ethidium bromide staining (see results below).
   Results

Results are shown in FIG. 5

Discussion

Mung Bean endonuclease can be used in the assay but it is not as effective as exonuclease I in reducing the background signal in reactions without CIP.

Sensitivity of Method 2 for Alkaline Phosphatase Detection

Background

The concentration on alkaline phosphatase in the assay was tested. The lowest amount of alkaline phosphatase needed to de-phosphorylate the DNA and hence protect it from lambda exonuclease digestion was determined.

Method

1. pUC19 DNA was used at both 1 ng and 100 pg in this experiment.
2. The DNA was incubated with 1 μl of serial 10-fold dilutions of alkaline phosphatase from a 1/500 dilution to a 1/500 000 dilution of the enzyme. Control tubes without alkaline phosphatase were prepared.
3. After 1 hr at 37° C. the reactions were incubated with lambda exonuclease and then exonuclease I as previously described.
4. PCR and agarose gel electrophoresis was as described.
   Results

Results are shown in FIG. 6

Discussion

It can be seen above that even 1 μl of the lowest concentration of alkaline phosphatase used in the assay ie1/500,000 is sufficient to act on the template DNA and protect it from nuclease such that it can be detected by PCR.

Method 2 Performed with an Immobilized Alkaline Phosphatase-Antibody Conjugate

Background

After optimizing the alkaline phosphatase detection assay in solution it was necessary to adapt this to a micro-well format. Hepatitis C was chosen as a model system and the viral core protein was used as the captured antigen by immobilized anti-HCV monoclonal antibody followed by the binding of an anti-core polyclonal antibody-alkaline phosphatase conjugate. Detection of alkaline phosphatase (CIP) and hence core protein (core) was determined by the assay described above. A schematic representation of the method is outlined in FIG. 7A.

Method

1. An anti-HCV core polyclonal antibody (Biodesign) was conjugated to maleimide activated alkaline phosphatase (EZ-Link kit, PIERCE).
2. 200 ng HCV monoclonal antibody (Biodesign) was bound to TopYield 8-well strips (NUNC) by adsorption at 37° C. for 1 hr.
3. Wells were washed and then blocked with 3% BSA in TBS
4. After washes, 200 ng HCV core antigen was added for 1 hr.
5. Antibody-alkaline phosphatase conjugate was then added at various dilutions (or not at all) and incubated for 1 hr at 37° C.
6. Wells were washed and pUC19 DNA added in 1×CIP reaction buffer to each well for 1 hr at 37° C.
7. 10 μl were then removed from each well and the protocol followed as described in Method 2 step 2 above.
   Results

Results are shown in FIG. 7B.

Discussion

This demonstrates that the invention can be used to convert an immuno protocol to a nucleic acid amplification detection format. An antibody-alkaline phosphatase conjugate, at an optimal concentration of 1/100 dilution (approximately 100 ng per micowell) in this example could be used to detect the HCV core antigen.

The Reduction of Background Signal Observed in Method 1

Background

Measures have been taken to improve the DNA template in order to reduce the level of background in the method. Background PCR signal is due to incomplete digestion of substrate by nucleases, probably due to non-phosphorylated template even in the absence of alkaline phosphates. In this example the preparation of a new template is described.

Method

1. A 285 bp ds 5′ phosphorylated DNA substrate for the enzyme was generated from the pUC19 (New England Biolabs, Beverly, Mass., USA) DNA template using a standard PCR process with the following 5′-phosphorylated primers (MWG Biotech, Milton Keynes, UK):

| | |
|---|---|
| PS1, | |
| 5′-P-GGCGAAAGGGGGATGTGCTGCAAGG-3′ | (SEQ ID NO:1) |
| and | |
| PAS1, | |
| 5′-P-GTGAGCGCAACGCAATTAATGTGAG-3′ | (SEQ ID NO:2) |
| and | |

Taq polymerase supplied by Qiagen Ltd, Crawley, UK. The use of “dual gel” purified oligonucleotides ensures a high percentage of phosphorylated primers.
2. Agarose gel purification of the DNA fragment following PCR and subsequent treatment of the DNA with T4 polynucleotide kinase (New England Biolabs, Beverly, Mass., USA) were additional measures performed to ensure minimal non-phosphorylated fragment.
3. Serial 10-fold dilutions of calf intestinal alkaline phosphatase (New England Biolabs, Beverly, Mass., USA) were prepared and incubated with the ds DNA substrate for 1 hour at 37° C. in Buffer 3 (from the enzyme supplier). 0.5 Units of lambda exonuclease (New England Biolabs, Beverly, Mass., USA) were then added and incubation was continued for a further 1 hr. 5 μl of each reaction mix were then amplified in a standard ‘detection’ PCR process using the PS1 and PAS1 primers. The amplification was carried out using an initial denaturation step at 94° C. (5 min) and subsequently 25 thermal cycles of the following profile: 94° C. (30 sec), 62° C. (30 sec), 72° C. (30 sec).
   Results

FIG. 8 shows the PCR products from the ‘detection’ step, separated by agarose gel electrophoresis and stained with ethidium bromide. Visual inspection indicates a detection limit of less than 10⁻¹¹ Units of alkaline phosphatase, which is approximately equivalent to 10×10⁻¹⁸ g, or as few as 60 molecules of AP in our assay.

Discussion

These results demonstrate that this methodology can be used to detect and quantify alkaline phosphatase activity with very high sensitivity. As a comparison, the detection limit when using pNPP colorimetric substrate (Sigma, Poole, UK) was determined to be 10⁻⁴ Units of alkaline phosphatase.

Increasing Sensitivity of Method 1 for Alkaline Phosphatase Detection

Background

Several important changes to method 1, especially when detecting AP-antibody conjugates, have been made to increase the sensitivity further. Important steps in the protocol include a high concentration of DNA template and performing the reaction at 55° C. instead of 37° C.

Method

1. Serial 10-fold dilutions of an anti-mouse IgG alkaline phosphatase conjugate (Sigma, Poole, UK) were prepared and incubated with the ds DNA substrate.
2. The DNA was at a final concentration in the reaction of 1 ng/ul.
3. The reaction was performed at 55° C. for 1 hour at in Buffer 3 (from the enzyme supplier).
4. 0.5 Units of lambda exonuclease (New England Biolabs, Beverly, Mass., USA) were then added and incubation was continued for a further 1 hr.
5. 2 μl of each reaction mix were then amplified in a standard ‘detection’ PCR process using the PS1 and PAS1 primers.
   Results

Detection limit can be seen to be less than 10⁻¹⁰ dilution of anti-mouse IgG-AP conjugate.

Discussion

Results show that the method for AP conjugate detected is very sensitive. The method described exhibited a detection limit over 100,000 times lower than the standard colorimetric pNPP substrate. This level of detection was comparable with that seen for free AP.

### REFERENCES

1. Compton. Nucleic acid sequence-based amplification. Nature 7; 350(6313): p91-92 (March 1991)
2. Fahy et al. Self-sustained sequence replication (3SR): an isothermal transcription-based amplification system alternative to PCR. PCR Methods Appl. 1(1): p25-33 (August) 1991
3. Lorentz; Continuous monitoring of prostatic acid phosphatase using self-inducing substrates. Clin Chim Acta.; 326 (1-2): p69-80 (December 2002).
4. Maldonado et al. Extremely high levels of alkaline phosphatase in hospitalized patients. J Clin Gastroenterol. 27(4): p342-345 (December 1998).
5. Wiwanitkit. High serum alkaline phosphatase levels, a study in 181 Thai adult hospitalized patients. BMC Family Practice. 2(1): 2 (July 2002).
6. Vogt, V. M. Purification and further properties of single-strand-specific nuclease from Aspergillus oryzae. Eur. J. Biochem. 33: p192-200 (1973)
7. Holland et al; Detection of specific polymerase chain reaction product by utilising the 5′-3′ exonuclease activity of Thermus aquaticus DNA polymerase; Proc. Natl. Acad. Sci. USA 88, 7276-7280 (1991).
8. Gelmini et al. Quantitative polymerase chain reaction-based homogeneous assay with flurogenic probes to measure C-Erbb-2 oncogene amplification. Clin. Chem. 43, 752-758 (1997).
9. Livak et al. Towards fully automated genome wide polymorphism screening. Nat. Genet. 9, 341-342 (1995)
10. Tyagi & Kramer. Molecular beacons—probes that fluoresce upon hybridization. Nat. Biotechnol. 14, 303-308 (1996).
11. Tyagi et al. Multicolor molecular beacons for allele discrimination. Nat. Biotechnol. 16, 49-53 (1998).
12. David Whitcombe, Jane Theaker, Simon P. Guy, Tom Brown, Steve Little. Detection of PCR products using self-probing amplicons and fluorescence; Nature Biotechnology 17, 804-807 (1 Aug. 1999) 11.

1. A method of detecting an enzyme in a sample, which enzyme is capable of adding or removing a chemical moiety to or from a nucleic acid molecule, thereby conferring altered sensitivity of the nucleic acid molecule in a subsequent process, the method comprising:
allowing the sample to be tested for the presence of the enzyme to interact with the nucleic acid molecule; and
testing for interaction of the enzyme with the nucleic acid molecule by detecting the altered sensitivity of the nucleic acid molecule caused by the enzyme.

2. A method according to claim 1 wherein the enzyme is a phosphatase capable of removing terminal phosphates from a nucleic acid molecule.

3. A method according to claim 1 wherein the enzyme is alkaline phosphatase.

4. A method according to claim 3 wherein the alkaline phosphatase is any of serum alkaline phosphatase, calf intestinal phosphatase (CIP), bacterial alkaline phosphatase (BAP) or shrimp alkaline phosphatase.

5. A method according to claim 1 wherein the enzyme being detected is prostatic acid phosphatase.

6. A method according to claim 1 wherein the altered sensitivity of the nucleic acid molecule that is detected is protection of the nucleic acid molecule from nuclease digestion.

7. A method according to claim 6 wherein the detection step is carried out by incubating the sample with nuclease enzymes and testing for the presence or absence of the nucleic acid molecule following incubation with nuclease enzymes.

8. A method according to claim 6 wherein the nuclease used to distinguish between nucleic acid molecules whose sensitivity in a subsequent process has been changed by enzyme activity in the sample and those whose sensitivity has not by digesting unchanged nucleic acid molecules comprises an exonuclease or both an endonuclease and an exonuclease or two complementary exonucleases.

9. A method according to claim 8 wherein the exonuclease used is lambda exonuclease.

10. A method according to claim 9 wherein the complementary exonuclease used is exonuclease I or the endonuclease used is mung bean endonuclease.

11. A method according to claim 1 wherein the nucleic acid molecule is blunt ended.

12. A method according to claim 1 wherein the nucleic acid molecule comprises dsDNA.

13. A method according to claim 1 wherein the nucleic acid molecule is phosphorylated at both 5′ ends.

14. A method according to claim 1 wherein the nucleic acid molecule is phosphorylated at a single 5′ end.

15. A method according to claim 1 wherein the nucleic acid molecule is produced by a DNA amplification technique using phosphorylated primers.

16. A method according to claim 1 wherein the nucleic acid is treated with a kinase enzyme.

17. A method according to claim 1 wherein the nucleic acid molecule is produced from a plasmid.

18. A method according to claim 17 wherein the plasmid comprises one of pUC derivatives and pBR322.

19. A method according to claim 17 wherein the plasmid is cleaved using restriction enzymes to produce blunt ended linear nucleic acid molecule(s).

20. A method according to claim 1 for detection of a phosphatase comprising the substeps of:
a) adding to the sample a nucleic acid molecule which comprises blunt ended dsDNA which is phosphorylated at one 5′ end only
b) incubating under conditions which permit phosphatase activity
c) adding lambda exonuclease and mung bean endonuclease or exonuclease I to the sample and allowing incubation with these enzymes; and
d) detecting the altered sensitivity of the nucleic acid molecule, measured as the presence or absence of the nucleic acid molecule.

21. A method according to claim 10 wherein the mung bean endonuclease or exonuclease I is at a concentration low enough to substantially prevent dsDNA digestion activity.

22. A method according to claim 1 for detection of a phosphatase comprising the substeps of:
a) adding to the sample a nucleic acid molecule which comprises blunt ended dsDNA which is phosphorylated at both 5′ ends
b) incubating under conditions which permit phosphatase activity
c) adding lambda exonuclease to the sample and allowing incubation with this enzyme; and
d) detecting the altered sensitivity of the nucleic acid molecule, measured as the presence or absence of the nucleic acid molecule.

23. A method according to claim 1 wherein the altered sensitivity of the nucleic acid molecule is detected using nucleic acid amplification techniques.

24. A method according to claim 23 wherein the nucleic acid amplification technique used is selected from PCR, Rolling Circle Amplification, NASBA, 3SR and TMA.

25. A method according to claim 23 wherein the products of amplification are detected using real-time techniques.

26. A method according to claim 25 wherein the real-time technique consists of any one of Taqman® system, Molecular beacons system and Scorpion probe system.

27. A method according to claim 1 wherein the enzyme is one which is used for detection of an antigen/analyte in an immunoassay.

28. A method according to claim 27 wherein the enzyme is attached, either directly or indirectly, to an antibody which is used in detection of an antigen/analyte.

29. A method according to claim 28 wherein the antibody is a primary antibody.

30. A method according to claim 28 wherein the antibody is a secondary antibody.

31. A method according to claim 1 wherein the method is carried out in a single tube.

32. A method according to claim 1 wherein the enzyme being detected is associated with an infectious agent, whereby detection of altered sensitivity of the nucleic acid molecule indicates the presence of the infectious agent.

33. A method according to claim 32 wherein the infectious agent is Aspergillus or Staphyloccocus species.

34. A method according to claim 1 for detection of a phosphatase enzyme associated with an infectious agent comprising the substeps of:
a) capture and separation of the infectious agent-specific phosphatase via a specific antibody
a) adding to the separated phosphatase a nucleic acid molecule which comprises blunt ended dsDNA which is phosphorylated at both 5′ ends
b) incubating under conditions which permit phosphatase activity
c) adding lambda exonuclease to the sample and allowing incubation with this enzyme; and
d) detecting the altered sensitivity of the nucleic acid molecule, measured as the presence or absence of the nucleic acid molecule, whereby detection of altered sensitivity indicates the presence of the infectious agent.

35. A method of diagnosing prostate cancer in a mammalian subject comprising
allowing a sample obtained from the subject under test to be tested for the presence of prostatic acid phosphatase (PAP) to interact with a nucleic acid molecule; and
testing for interaction of PAP with the nucleic acid molecule by detecting the altered sensitivity in the nucleic acid molecule caused by PAP in a downstream process, whereby the presence of prostatic acid phosphatase (PAP) in the sample is taken as an indication that the subject has prostate cancer.

36. A method of diagnosing a disease associated with elevated serum alkaline phosphatase levels in a mammalian subject comprising
allowing a sample obtained from the subject to be tested for the presence of serum alkaline phosphatase to interact with a nucleic acid molecule; and
testing for interaction of serum alkaline phosphatase with the nucleic acid molecule by detecting an altered sensitivity in the nucleic acid molecule caused by serum alkaline phosphatase, whereby the presence of serum alkaline phosphatase in the sample is taken as an indication that the subject has a disease associated with elevated serum alkaline phosphatase levels.

37. A method according to claim 36 wherein the method is used to diagnose any one of sepsis, AIDS, malignancies, obstructive biliary diseases, infiltrative liver diseases, sepsis and cholangiocarcinoma.

38. A method according to claim 35 wherein the method is carried out in vitro.

39. A method according to claim 35 which further comprises obtaining the sample from the subject.

40. A method according to claim 35 wherein the subject is a human.

41. A kit for detecting an enzyme capable of adding or removing a chemical moiety to or from a nucleic acid molecule, which thereby confers altered sensitivity of the nucleic acid molecule in a subsequent process, comprising:
a nucleic acid molecule which is capable of being acted upon by the enzyme; and
means for detecting the altered sensitivity of the nucleic acid molecule in the subsequent process.

42. A kit according to claim 41 wherein the nucleic acid molecule comprises dsDNA.

43. A kit according to claim 41 wherein the nucleic acid molecule is blunt ended.

44. A kit according to claim 41 wherein the nucleic acid molecule is phosphorylated at one or both 5′ ends.

45. A kit according to claim 41 wherein the nucleic acid molecule comprises a plasmid which can be cut using restriction enzymes to give blunt ended dsDNA which is phosphorylated at both 5′ ends.

46. A kit according to claim 45 wherein the plasmid is a pUC derivative or pBR322.

47. A kit according to claim 45 further including the restriction enzymes necessary to cut the plasmid.

48. A kit according to claim 41 wherein the means for measuring the altered sensitivity of the nucleic acid molecule includes an exonuclease and/or an endonuclease or a complementary exonuclease which digests the nucleic acid molecule if the nucleic acid molecule has not been modified by the enzyme.

49. A kit according to claim 48 wherein the endonuclease comprises mung bean endonuclease or the complementary exonuclease comprises exonuclease I.

50. A kit according to claim 48 wherein the exonuclease comprises lambda exonuclease.

51. A kit according to claim 41 wherein the enzyme being detected is alkaline phosphatase.

52. A kit according to claim 41 wherein the means for measuring the altered sensitivity of the nucleic acid molecule requires nucleic acid amplification.

53. A kit according to claim 41 wherein the kit further includes reagents necessary for nucleic acid amplification.

54. A kit according to claim 52 wherein the nucleic acid amplification step is selected from PCR, NASBA, Rolling circle amplification, 3SR and TMA.

55. A kit according to claim 52 wherein the kit further comprises probes and reagents necessary for real-time detection of nucleic acid amplification products.

56. A kit according to claim 55 wherein the real-time detection method is selected from Taqman® system, Molecular beacons system and Scorpion probe system.

57. A kit according to claim 41 further comprising an antibody selective for an infectious agent-specific phosphatase.

58. A kit according to claim 57 wherein the infectious agent is Aspergillus or Staphyloccocus species.


SEQUENCE LISTING (2 sequences)

SEQ ID NO: 1 - DNA, 25 bp, Artificial sequence
  5′ phosphorylated primer
ggcgaaagggggatgtgctgcaagg

SEQ ID NO: 2 - DNA, 25 bp, Artificial sequence
  5′ phosphorylated primer
gtgagcgcaacgcaattaatgtgag